(19) United States
(12) Patent Application Publication
(10) Pub. No.: US 2023/0290493 A1
(43) Pub. Date: Sep. 14, 2023

(54) MEDICAL DEVICE DIAGNOSTICS AND ALERTING

(71) Applicant: BOARD OF REGENTS OF THE UNIVERSITY OF NEBRASKA, Lincoln, NE (US)

(72) Inventors: William Owen Redwood, Omaha, NE (US); Jeff Dunn, Omaha, NE (US); Riley Hester, Omaha, NE (US); Ian Trent, Omaha, NE (US); Eric Wright, Omaha, NE (US); Brad Miller, Omaha, NE (US); Orly Tauil, Omaha, NE (US)

(21) Appl. No.: 18/007,217

(22) PCT Filed: Jun. 24, 2021
(86) PCT No.: PCT/US2021/038805

§ 371 Date: Jan. 27, 2023

Related U.S. Application Data
(60) Provisional application No. 63/071,158, filed on Aug. 27, 2020.

(51) Int. Cl. G16H 40/40

(52) U.S. Cl. CPC G16H 40/40

(57) ABSTRACT

Methods and systems for medical device diagnostics and reporting. One system including an electronic processor configured to receive a new medical device dataset associated with a medical device. The electronic processor is also configured to determine an operation classification of the medical device using a model utilizing training information. The training information includes a plurality of archived medical device datasets and an associated operation classification for each of the plurality of archived medical device datasets. The electronic processor is also configured determine an operation status of the medical device based on the operation classification. The electronic processor is also configured to generate and provide a notification based on at least the operation status of the medical device.

### CROSS-REFERENCE TO RELATED APPLICATIONS

This application claims priority to U.S. Provisional Application No. 63/071,158 filed on Aug. 27, 2020, which is incorporated fully herein by reference.

### GOVERNMENT FUNDING

The subject matter of this invention was made with Government support under contract FA8750-16-C-0178, subcontract PO-0017642 awarded by Defense Advanced Research Projects Agency (DARPA)/Air Force Research Labs (AFRL). The Government has certain rights to this invention.

### FIELD

Embodiments described herein relate to medical device diagnostics and alerting, and, more particularly, to diagnosing whether a medical device is functioning properly or is subject to a cyber-attack.

### BACKGROUND

Biomedical devices, such as pacemakers, insulin pumps, health monitors, cardiac defibrillators, spinal cord neurostimulators, transcutaneous electrical nerve simulators, and the like, are generally configured to monitor a health condition of an associated user, perform an operation associated with a health condition of the associated user, or a combination thereof. These biomedical devices may be controlled remotely. Therefore, these biomedical devices can be subject to various cyber-attacks. Accordingly, when patients are in jeopardy, it is difficult to determine whether cyber-attacks against the patient's life-critical biomedical devices are involved.

### SUMMARY

Embodiments described herein relate to methods and systems of medical device diagnostics and alerting for determining whether a medical device is functioning properly or is subject to a cyber-attack (or had been the subject of a cyber-attack). Accordingly, embodiments described herein enable first responders and other medical professionals to diagnose implantable or wearable medical devices to determine whether a device has been or is malfunctioning such that it may be the subject of a cyber-attack. Embodiments collect medical device datasets, logs, and alerts from medical devices and translates nuanced cyber-factors included in the data into actionable information or instructions for clinical users.

Accordingly, embodiments described herein address the problem that when patients are in jeopardy, current approaches or systems cannot determine whether a device malfunction or cyber-attack against the patient's life-critical biomedical device(s) was involved. As one example, an EMT arrives at the scene of a patient having a cardiac event. The standard procedure may be to administer CPR. The EMT is alerted that the patient may have a pacemaker implanted on their person. However, utilizing the methods and systems described herein, the EMT may scan the implanted device to determine whether the device was the subject of a malfunction or cyber-attack. Therefore, the embodiments described herein enable medical care providers, first responders (for example, police, fire, EMT, and the like), and the like to utilize the systems and methods described herein to interface with medical devices (either external or implantable) to ascertain the overall health and functionality of the medical device, such that a device malfunction or a malicious actor impacting the devices through cyber means may be detected or ruled out.

Accordingly, embodiments described herein provide systems and methods for medical device diagnostics and alerting. For example, one embodiment provides a system for medical device diagnostics and alerting. The system includes an electronic processor configured to receive a new medical device dataset associated with a medical device. The electronic processor is also configured to determine an operation classification of the medical device using a model developed with machine learning using training information, the training information including a plurality of archived medical device datasets and an associated operation classification for each of the plurality of archived medical device datasets. The electronic processor is also configured to determine an operation status of the medical device based on the operation classification. The electronic processor is also configured to generate and provide a notification based on at least the operation status of the medical device.

Another embodiment provides a medical device diagnostics and alerting. The method includes receiving a new medical device dataset associated with a medical device. The method also includes determining, with an electronic processor, an operation classification of the medical device using a model developed with machine learning. The method also includes determining an operation status of the medical device based on the operation classification. The method also includes in response to determining the operation status of the medical device to be an abnormal operation status or a suspicious operation status, generating and providing, with the electronic processor, a notification based on at least the operation status of the medical device.

Yet another embodiment provides a non-transitory computer readable medium including instructions that, when executed by an electronic processor, causes the electronic processor to execute a set of functions. The set of functions includes receiving a new medical device dataset associated with a medical device. The set of functions also includes determining an operation classification of the medical device using a model developed with machine learning using training information, the training information including a plurality of archived medical device datasets and an associated operation classification for each of the plurality of archived medical device datasets. The set of functions also includes determining an operation status of the medical device based on the operation classification. The set of functions also includes, in response to determining the operation status of the medical device to be an abnormal operation status or a suspicious operation status, generating and providing a notification based on at least the operation status of the medical device.

Other aspects of the embodiments will become apparent by consideration of the detailed description and accompanying drawings.

### BRIEF DESCRIPTION OF THE DRAWINGS

FIG. 1 illustrates a system for medical device diagnostics and alerting according to some embodiments.

FIG. 2 illustrates a server included in the system of FIG. 1 according to some embodiments.

FIG. 3 illustrates a medical device included in the system of FIG. 1 according to some embodiments.

FIG. 4 is a flowchart illustrating a method for medical device diagnostics and alerting using the system of FIG. 1 according to some embodiments.

FIG. 5 is an example communication diagram illustrating the communication between components of the system 100 according to some embodiments.

Other aspects of the embodiments described herein will become apparent by consideration of the detailed description.

### DETAILED DESCRIPTION

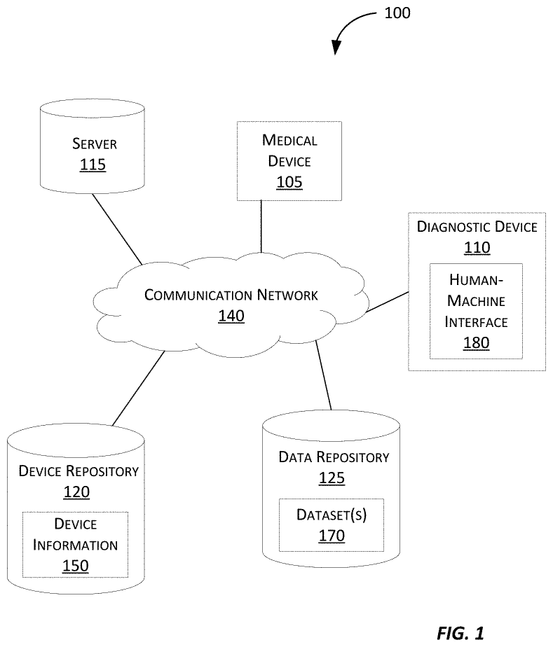

FIG. 1 schematically illustrates a system 100 for medical device diagnostics and alerting according to some embodiments. The system 100 includes a medical device 105, a diagnostic device 110, a server 115, a device repository 120, and a data repository 125. In some embodiments, the system 100 includes fewer, additional, or different components than illustrated in FIG. 1. For example, the system 100 may include multiple medical devices 105, diagnostic devices 110, servers 115, device repositories 120, data repositories 125, or a combination thereof. Additionally, in some embodiments, one or more components of the system 100 may be distributed among multiple devices, servers, or databases, combined into a single device, server, or database. As one example, the device repository 120 and the data repository 125 may be combined into a single database.

The medical device 105, the diagnostic device 110, the server 115, the device repository 120, and the data repository 125 communicate over one or more wired or wireless communication networks 140. Portions of the communication network 140 may be implemented using a wide area network (“WAN”), such as the Internet, a local area network (“LAN”), such as a Bluetooth™ network or Wi-Fi, and combinations or derivatives thereof. Alternatively or in addition, in some embodiments, components of the system 100 communicate directly as compared to through the communication network 140. Also, in some embodiments, the components of the system 100 communicate through one or more intermediary devices not illustrated in FIG. 1.

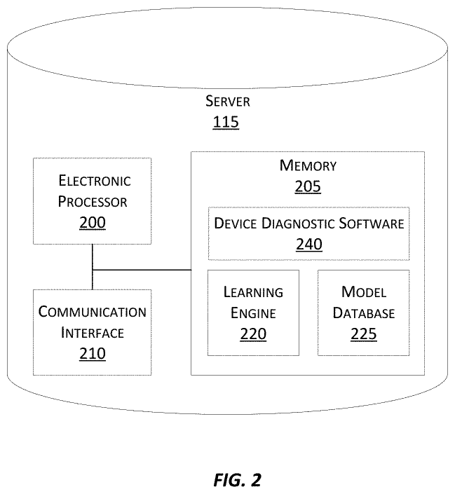

As illustrated in FIG. 2, the server 115 includes an electronic processor 200, a memory 205, and a communication interface 210. The electronic processor 200, the memory 205, and the communication interface 210 communicate wirelessly, over one or more communication lines or buses, or a combination thereof. The server 115 may include additional, fewer, or different components than those illustrated in FIG. 2 in various configurations. The server 115 may also perform additional functionality other than the functionality described herein. Also, the functionality (or a portion thereof) described herein as being performed by the server 115 may be distributed among multiple devices, such as multiple servers or devices included in a cloud service environment. In addition, in some embodiments, the diagnostic device 110 may be configured to perform all or a portion of the functionality described herein as being performed by the server 115.

The electronic processor 200 includes a microprocessor, an application-specific integrated circuit (“ASIC”), or another suitable electronic device for processing data. The memory 205 includes a non-transitory computer readable medium, such as read-only memory (“ROM”), random access memory (“RAM”) (for example, dynamic RAM (“DRAM”), synchronous DRAM (“SDRAM”), and the like), electrically erasable programmable read-only memory (“EEPROM”), flash memory, a hard disk, a secure digital (“SD”) card, another suitable memory device, or a combination thereof. The electronic processor 200 is configured to access and execute computer-readable instructions (“software”) stored in the memory 205. The software may include firmware, one or more applications, program data, filters, rules, one or more program modules, and other executable instructions. For example, the software may include instructions and associated data for performing a set of functions, including the methods described herein.

For example, as illustrated in FIG. 2, the memory 205 may store a learning engine 220 and a model database 225. In some embodiments, the learning engine 220 develops a model using one or more machine learning functions. Machine learning functions are generally functions that allow a computer application to learn without being explicitly programmed. In particular, a computer application performing machine learning functions (sometimes referred to as a learning engine) is configured to develop an algorithm based on training data or training information. For example, to perform supervised learning, the training data includes example inputs and corresponding desired (for example, actual) outputs, and the learning engine progressively develops a model that maps inputs to the outputs included in the training data. Machine learning may be performed using various types of methods and mechanisms including but not limited to decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using one or more of these approaches, a computer program may ingest, parse, and understand data and progressively refine models for data analytics, including medical device diagnostics and alerting.

Accordingly, the learning engine 220 (as executed by the electronic processor 200) may perform machine learning using training data to develop a model that maps a medical device dataset to an operation classification. The training data may include, for example, medical device datasets and their associated operation classifications. For example, the learning engine 220 may identify one or more unique characteristics, trends, or defects of the medical device dataset (for example, anomalies or outliers included in the medical device dataset, features indicating malicious activity or tampering, features indicating a malfunction, and the like) and develop a model that maps the one or more unique characteristics, trends, or defects to a particular operation classification, such as abnormal operation, suspicious operation, or normal operation. Accordingly, when a subsequent medical device dataset is received, the developed model may be used to determine an operation classification for that subsequent medical device dataset. In other words, the model, once trained, analyzes a medical device dataset to identify one or more characteristics, trends, or defects in the medical device dataset and assigns the medical device dataset an operation classification based on any detected characteristics, trends, or defects. As described in more detail below, in some embodiments, the model is applied to a medical device dataset (or a new medical device dataset) at the point of data acquisition from, for example, the medical device 105 with the diagnostic device 110.

Models generated by the learning engine 220 may be stored in the model database 225. As illustrated in FIG. 2, the model database 225 is included in the memory 205 of the server 115. In some embodiments, the model database 225 may be located external to the server 115. In such embodiments, the server 115 may communicate with and access data from the model database 225 directly or through one or more of the communication network(s) 140. Also, in some embodiments, the model database 225 may be included in or part of the device repository 120, the data repository 125, the medical device 105, the diagnostic device 110, or a combination thereof, which the server 115 may similarly access.

As illustrated in FIG. 2, the memory 205 may also store device diagnostic software 240. The device diagnostic software 240 is a software application executable by the electronic processor 200. As described in more detail below, the device diagnostic software 240, when executed by the electronic processor 200, performs medical device diagnostics and reporting. As one example, the device diagnostic software 240 may receive or access a medical device dataset and determine an operation classification for the medical device 105 using one or more models included in the model database 225. Alternatively or in addition, in some embodiments, the device diagnostic software 240 generates and provides information or instructions (for example, as a notification) based on the medical device dataset, the operation classification for the medical device associated with the medical device dataset, or a combination thereof.

The communication interface 210 allows the server 115 to communicate with devices external to the server 115. For example, as illustrated in FIG. 1, the server 115 may communicate with the medical device 105, the diagnostic device 110, the device repository 120, the data repository 125, or a combination thereof through the communication interface 210. In particular, the communication interface 210 may include a port for receiving a wired connection to an external device (for example, a universal serial bus (“USB”) cable and the like), a transceiver for establishing a wireless connection to an external device (for example, over one or more communication networks 140, such as the Internet, a LAN, a WAN, and the like), or a combination thereof.

As illustrated in FIG. 1, the device repository 120 stores device information 150. Device information 150 may include a plurality of device manuals, where each device manual includes information associated with a particular medical device (for example, the medical device 105). A device manual may be, for example, a manufacturer's manual for an associated medical device, a user's guide for an associated medical device, or the like. Alternatively or in addition, in some embodiments, the device information 150 includes a listing of diagnostic codes for a particular medical device (for example, the medical device 105). In some embodiments, the device information 150 stored in the device repository 120 is used as (or is part of) the training information for the models stored in the model database 225.

Accordingly, the device database 120 provides for the storage and retrieval of device information 150. In some embodiments, the device information 150 may be stored within a plurality of databases, such as within a cloud service. Although not illustrated in FIG. 1, the device repository 120 may include components similar to the server 115, such as an electronic processor, a memory, a communication interface, and the like. For example, the device repository 120 may include a communication interface configured to communicate (for example, receive data and transmit data) over the communication network 140.

The data repository 125 stores one or more datasets 170 (referred to herein collectively as “the datasets 170” and individually as “the dataset 170”). A dataset 170 may also be referred to herein as a medical device dataset. In some embodiments, each of the datasets 170 stored in the data repository 125 correspond with at least one medical device. In other words, a dataset 170 includes data or readings associated with at least one corresponding medical device. As one example, the dataset 170 may include historical or previously collected medical data or readings from the medical device 105. As another example, the dataset 170 may include medical data or readings from an additional or different medical device, such as a medical device associated with another user. Accordingly, in some embodiments, the datasets 170 stored in the data repository 125 is a collection of aggregated data from one or more medical devices (for example, a plurality of archived medical device datasets). In some embodiments, the datasets 170 stored in the data repository 125 are used as the training information for the models stored in the model database 225. In such embodiments, the data repository 125 also stores an associated operation classification for one or more of the datasets 170.

Accordingly, the data repository 125 provides for the storage and retrieval of the datasets 170 corresponding to one or more medical devices (for example, the medical device 105, another user's medical device, and the like). Although not illustrated in FIG. 1, the data repository 125 may include components similar to the server 115, such as an electronic processor, a memory, a communication interface and the like. For example, the data repository 125 may include a communication interface configured to communicate (for example, receive data and transmit data) over the communication network 140.

In some embodiments, the device repository 120 and the data repository 170 are combined to form a single repository that stores the device information 150, the datasets 170, or a combination thereof. Additionally, in some embodiments, the device repository 120 and the data repository 170 are combined into multiple individual repositories, where each individual repository stores device information 150 associated with a particular medical device and stores datasets 170 associated with that particular medical device. Alternatively or in addition, the datasets 170, the device information 150, or a combination thereof may be stored within a plurality of databases or repositories, such as within a cloud service.

The medical device 105 is configured to monitor a health condition of an associated user, perform an operation associated with a health condition of the associated user, or a combination thereof. The medical device 105 may be, for example, a pacemaker, an insulin pump, a health monitor, a cardiac defibrillator, a spinal cord neurostimulator, a transcutaneous electrical nerve simulator, and the like. Accordingly, in some embodiments, the medical device 105 is implanted within a user's body. However, in other embodiments, the medical device 105 is external to the user's body, such as a wearable medical device.

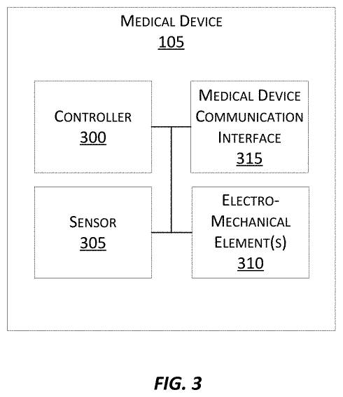

As illustrated in FIG. 3, the medical device 105 includes a controller 300, a sensor 305, one or more electro-mechanical (“EM”) elements 310 (referred to collectively as “the EM elements 310 and individually as “the EM element 310”), and a medical device communication interface 315. The controller 300, the sensor 305, the EM elements 310, and the medical device communication interface 315 communicate wirelessly, over one or more communication lines or buses, or a combination thereof. The medical device 105 may include additional, fewer, or different components than those illustrated in FIG. 3 in various configurations. As one example, the medical device 105 may include one or more energy sources configured to power the medical device 105 (or the components thereof). As another example, the medical device 105 may include multiple sensors 305, controllers 300, or a combination thereof. The medical device 105 may also perform additional functionality other than the functionality described herein. Also, the functionality (or a portion thereof) described herein as being performed by the medical device 105 may be distributed among multiple devices, such as multiple networked medical devices 105.

The sensor 305 collects data related to a health condition of the user (for example, a medical device dataset). The sensor 305 may include, for example, a force sensor, a strain sensor, an image sensor, a vibration sensor, a photo optic sensor, a piezoelectric sensor, a pressure sensor, a position sensor, a temperature sensor, a blood glucose sensor, an electrocardiogram (“ECG”) sensor, a motion sensor, an inertial sensor, and the like. The data collected by the sensor 305 may be stored in a memory (not shown) of the medical device 105. The EM element 310 is configured to perform an action or operation related to a health condition of a user. The EM element 310 may include, for example, a valve, an actuator, a pulse generator, an electrode, a reservoir, a motor, a pump, or the like. In some embodiments, the controller 300 controls one or more of the EM elements 310 based on data collected by the sensor 305. As one example, the controller 300 may access the collected data and determine whether to perform an action or operation based on the collected data. Alternatively or in addition, the controller 300 may determine what action or operation to perform, how to perform that action or operation based on the collected data, or a combination thereof. When the controller 300 determines that a particular action should be performed, the controller 300 may transmit a control signal to a corresponding EM element 310. In response to receiving the control signal, the EM element 310 may perform the action or operation according to the control signal.

The medical device communication interface 315 allows the medical device 105 to communicate with devices external to the medical device 105. For example, as illustrated in FIG. 1, the medical device 105 may communicate with the server 115, the diagnostic device 110, the device repository 120, the data repository 125, or a combination thereof through the medical device communication interface 315. In particular, the medical device communication interface 315 may include a port for receiving a wired connection to an external device (for example, a USB cable and the like), a transceiver for establishing a wireless connection to an external device (for example, over one or more communication networks 140, such as the Internet, a LAN, a WAN, and the like), or a combination thereof. As one example, the medical device 105 may communicate one or more medical device datasets to the diagnostic device 110 through the medical device communication interface 315.

The diagnostic device 110 is a computing device and may include, for example, a desktop computer, a terminal, a workstation, a laptop computer, a tablet computer, a smart watch or other wearable, a smart television or whiteboard, or the like. Alternatively or in addition, the diagnostic device 110 may be a mobile communication device, such as a smart cellular device or phone. Although not illustrated, the diagnostic device 110 may include similar components as the server 115 (an electronic processor, a memory, and a communication interface). As seen in FIG. 1, the diagnostic device 110 may also include a human-machine interface 180 for interacting with a user. The human-machine interface 180 may include one or more input devices, one or more output devices, or a combination thereof. Accordingly, in some embodiments, the human-machine interface 180 allows a user to interact with (for example, provide input to and receive output from) the diagnostic device 110. For example, the human-machine interface 180 may include a keyboard, a cursor-control device (for example, a mouse), a touch screen, a scroll ball, a mechanical button, a display device (for example, a liquid crystal display (“LCD”)), a printer, a speaker, a microphone, or a combination thereof. In some embodiments, the human-machine interface 180 includes a display device. The display device may be included in the same housing as the diagnostic device 110 or may communicate with the diagnostic device 110 over one or more wired or wireless connections. For example, in some embodiments, the display device is a touchscreen included in a laptop computer, a tablet computer, or a mobile communication device. In other embodiments, the display device is a monitor, a television, or a projector coupled to a terminal, desktop computer, or the like via one or more cables.

The diagnostic device 110 is configured to read (or receive) medical device datasets from medical devices (for example, the medical device 105) and provide information associated with the medical device datasets to a user of the diagnostic device 110. Additionally, in some embodiments, the diagnostic device 110 transmits the medical device datasets for external analysis by, for example, the server 115. In some embodiments, the diagnostic device 110 is able to distinguish the medical device 105 from other medical devices or equipment within the surrounding of or near by the medical device 105.

A user of the diagnostic device 110 may include, for example, a first responder (for example, a firefighter, a paramedic, or the like), a medical or clinical personal (for example, a treating physician, a nurse, a physician's assistant, or the like), another user (for example, a user of the medical device 105), or a combination thereof. As one example, the diagnostic device 110 may be a mobile communication device issued to a first responder. As another example, the diagnostic device 110 may be a mobile communication device of the user of the medical device 105, such as the user's cellphone. As yet another example, the diagnostic device 110 may be a computing device located within a medical clinic or hospital.

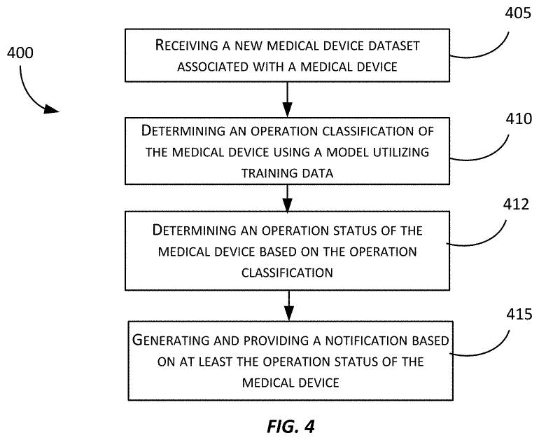

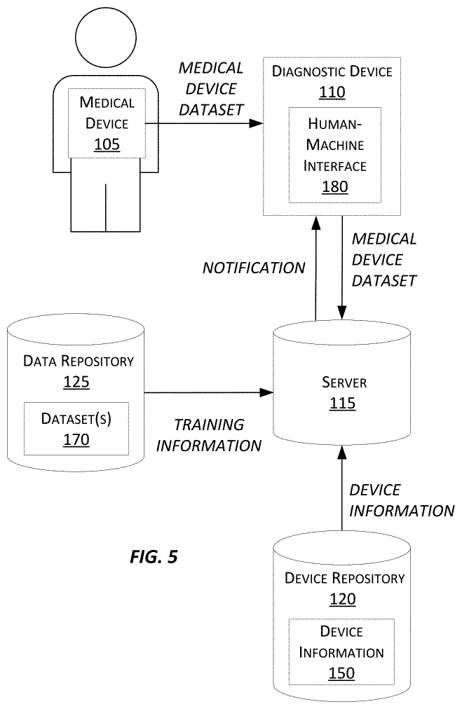

FIG. 4 illustrates a method 400 for medical device diagnostics and reporting using the system of FIG. 1 according to some embodiments. The method 400 is described herein as being performed by the server 115 and, in particular, the device diagnostic software 240 as executed by the electronic processor 200. However, as noted above, the functionality performed by the server 115 (or a portion thereof) may be performed by other devices, including, for example, the diagnostic device 110 (via an electronic processor executing instructions). The method 400 is described with reference to FIG. 5. FIG. 5 is an example communication diagram illustrating the communication between components of the system 100 according to some embodiments.

As illustrated in FIG. 4, the method 400 includes receiving, with the electronic processor 200, a new medical device dataset associated with the medical device 105 (at block 405), as illustrated in FIG. 5. The new medical device dataset may be medical data or readings collected by the medical device 105 (for example, by the sensor 305). In some embodiments, the electronic processor 200 receives the new medical device dataset directly from the medical device 105. However, in other embodiments, the electronic processor 200 receives the new medical device dataset from the diagnostic device 110, which originally received the new medical device dataset from the medical device 105. As described above, the diagnostic device 110 is configured to read (or receive) datasets from medical devices, such as the new medical device dataset from the medical device 105. In such embodiments, the diagnostic device 110 forwards (or transmits) the new medical device dataset to the server 115 (the electronic processor 200) for remote analytics and diagnostics. In some embodiments, the diagnostic device 110 automatically transmits the new medical device dataset to the server 115 in response to receiving the new medical device dataset. However, in other embodiments, the diagnostic device 110 transmits the new medical device dataset to the server 115 in response to a request, such as a manual request from a user of the diagnostic device 110.

After receiving the new medical device dataset (at block 405), the electronic processor 200 determines an operation classification of the medical device based on the new medical device dataset (at block 410). In some embodiments, the electronic processor 200 determines the operation classification using one or more models developed with machine learning using training information, such as one or more of the models stored in the model database 225. As noted above, the training information may include a plurality of archived medical device datasets (for example, the datasets 170 stored in the data repository 125, as seen in FIG. 5) and an associated operation classification for each of the plurality of archived medical device datasets. Accordingly, in such embodiments, the electronic processor 200 accesses the one or more models from the model database 225 and analyzes the new medical device dataset using the accessed one or more models (for example, applies the one or more models to the new medical device dataset).

The electronic processor 200 may then determine an operation status of the medical device 105 (at block 412). Each associated operation classification may indicate an operation status for an associated medical device. The operation status of the medical device 105 may include, for example, an abnormal operation status, a normal operation status, or a suspicious operation status. A normal operation status may indicate that the medical device 105 is operating or functioning normally. An abnormal operation status may indicate that the medical device 105 is not operating or functioning normally. As one example, the electronic processor 200 may determine the operation status to be an abnormal operation status when the medical device 105 is malfunctioning, such as a malfunction resulting from a fault experienced by a component of the medical device 105. A suspicious operation status may indicate that the medical device 105 was tampered with or subjected to a cyber-attack (or is currently being tampered with or subjected to a cyber-attack). As one example, the electronic processor 200 may determine the operation status to be a suspicious operation status when the medical device 105 was hacked.

As seen in FIG. 4, the electronic processor 200 may generate and provide a notification based on at least the operation status of the medical device 105 (at block 410). In some embodiments, the notification includes a set of instructions for treating a user of the medical device 105. As one example, when the electronic processor 200 determines that the operation status of the medical device 105 is an abnormal operation status, the electronic processor 200 may generate and provide a notification including an instruction to “Start reading patient vitals by another means due to a malfunction.” As another example, when the electronic processor 200 determines that the operation status of the medical device 105 is a suspicious operation status, the electronic processor 200 may generate and provide a notification including an instruction to “Please alert cyber security or IT team that device was tampered with.” Alternatively or in addition, the notification may include the operation status of the medical device 105. As one example, when the electronic processor 200 determines that the operation status of the medical device 105 is a normal operation status, the electronic processor 200 may generate and provide a notification indicating that the medical device 105 is exhibiting normal operation. In some embodiments, the notification includes information associated with a user of the medical device 105. Such information may include, for example, an identification of the user (for example, a name of the user), a characteristic of the user (for example, an age, a blood type, or the like), a health condition of the user, a medicine list of the user, and the like. Alternatively or in addition, the notification includes information associated with the medical device 105, such as information included in the device information 150 for the medical device 105. Alternatively or in addition, the notification may include the new medical device dataset, including raw data or readings of the new medical device dataset.

In some embodiments, the electronic processor 200 generates and provides the notification in response to determining the operation status of the medical device 105 is an abnormal operation status, a suspicious operation status, or a combination thereof. Accordingly, in such embodiments, the electronic processor 200 may generate and provide the notification in situations where the medical device 105 may have been tampered with (a suspicious operation status) or is malfunctioning (an abnormal operation status).

In some embodiments, the electronic processor 200 generates and provides the notification by transmitting the notification to the diagnostic device 110, as seen in FIG. 5. In such embodiments, the diagnostic device 110 may provide the notification to a user of the diagnostic device 110 in response to receiving the notification from the electronic processor 200. As one example, the diagnostic device 110 may display the notification via the human-machine interface 180 (for example, a display device of the diagnostic device 110). As another example, the diagnostic device 110 may verbally provide the notification via a microphone component of the diagnostic device 110.

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way, but may also be configured in ways that are not listed. Furthermore, some embodiments described herein may include one or more electronic processors configured to perform the described functionality by executing instructions stored in non-transitory, computer-readable medium. Similarly, embodiments described herein may be implemented as non-transitory, computer-readable medium storing instructions executable by one or more electronic processors to perform the described functionality. As used in the present application, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms such as first and second, top and bottom, and the like may be used herein solely to distinguish one entity or action from another entity or action without necessarily requiring or implying any actual such relationship or order between such entities or actions.

Various features and advantages of the embodiments are set forth in the following claims.

What is claimed is:

1. A system for medical device diagnostics and reporting, the system comprising:
an electronic processor configured to
receive a new medical device dataset associated with a medical device,
determine an operation classification of the medical device using a model utilizing training information, the training information including a plurality of archived medical device datasets and an associated operation classification for each of the plurality of archived medical device datasets,
determine an operation status of the medical device based on the operation classification, and
generate and provide a notification based on at least the operation status of the medical device.

2. The system of claim 1, wherein the operation status of the medical device includes at least one selected from a group consisting of an abnormal operation, a suspicious operation, and a normal operation.

3. The system of claim 1, wherein the electronic processor is configured to generate and provide the notification in response to determining the operation status of the medical device as an abnormal operation status or as a suspicious operation status.

4. The system of claim 1, wherein the notification includes a set of instructions for treating a user of the medical device.

5. The system of claim 1, wherein the electronic processor is configured to receive the new medical device dataset directly from the medical device.

6. The system of claim 1, wherein the electronic processor is configured to receive the new medical device dataset from a diagnostic device external to the medical device.

7. The system of claim 1, wherein the medical device is an implanted medical device.

8. The system of claim 1, wherein the electronic processor is configured to generate and provide the notification by transmitting the notification to a diagnostic device external to the medical device.

9. The system of claim 8, wherein the diagnostic device is configured to provide the notification via a human-machine interface of the diagnostic device.

10. A method of medical device diagnostics and alerting, the method comprising:
receiving a new medical device dataset associated with a medical device;
determining, with an electronic processor, an operation classification of the medical device using a model developed with machine learning;
determining an operation status of the medical device based on the operation classification; and
in response to determining the operation status of the medical device to be an abnormal operation status or a suspicious operation status, generating and providing, with the electronic processor, a notification based on at least the operation status of the medical device.

11. The method of claim 10, wherein determining the operation classification of the medical device includes determining the operation classification of the medical device using the model developed with machine learning using training information, wherein the training information includes a plurality of archived medical device datasets and an associated operation classification for each of the plurality of archived medical device datasets.

12. The method of claim 10, wherein generating and providing the notification includes generating and providing a set of instructions for treating a user of the medical device.

13. The method of claim 10, wherein receiving the new medical device dataset includes receiving the new medical device dataset directly from the medical device.

14. The method of claim 10, wherein receiving the new medical device dataset includes receiving the new medical device dataset from a diagnostic device external to the medical device.

15. The method of claim 10, wherein generating and providing the notification includes transmitting the notification to a diagnostic device external to the medical device, wherein the diagnostic device is configured to provide the notification via a human-machine interface of the diagnostic device.

16. A non-transitory, computer-readable medium storing instructions that, when executed by an electronic processor, perform a set of functions, the set of functions comprising:
receiving a new medical device dataset associated with a medical device;
determining an operation classification of the medical device using a model developed with machine learning using training information, the training information including a plurality of archived medical device datasets and an associated operation classification for each of the plurality of archived medical device datasets;
determining an operation status of the medical device based on the operation classification; and
in response to determining the operation status of the medical device to be an abnormal operation status or a suspicious operation status, generating and providing a notification based on at least the operation status of the medical device.

17. The computer-readable medium of claim 16, wherein generating and providing the notification includes generating and providing the notification including the operation status.

18. The computer-readable medium of claim 16, wherein generating and providing the notification includes generating and providing the notification including at least one selected from a group consisting of raw data included in the new medical device dataset, information associated with a user of the medical device, and device information from a device manual of the medical device.

19. The computer-readable medium of claim 16, wherein receiving the new medical device dataset includes receiving the new medical device dataset from a diagnostic device external to the medical device.

20. The computer-readable medium of claim 16, wherein generating and providing the notification includes transmitting the notification to a diagnostic device external to the medical device, wherein the diagnostic device is configured to provide the notification via a human-machine interface of the diagnostic device.
